(19) United States
(12) Patent Application Publication
(10) Pub. No.: US 2015/0231047 A1
(43) Pub. Date: Aug. 20, 2015

(54) EXOTHERMIC HAIR COLOR COMPOSITION

(71) Applicant: 6569048 Canada Inc., Dorval (CA)

(72) Inventors: Moty Cohen, Laval (CA); Dulce Cristina Almeida Da Silva, Montreal (CA)

(21) Appl. No.: 14/621,561

(22) Filed: Feb. 13, 2015

Related U.S. Application Data
(60) Provisional application No. 61/940,063, filed on Feb. 14, 2014.

(51) Int. Cl. A61K 8/46; A61K 8/23; A61K 8/34; A61Q 5/10; A61K 8/41

(52) U.S. Cl. CPC A61K 8/463; A61Q 5/10; A61K 8/415; A61K 8/345; A61K 8/23; A61K 8/41; A61K 2800/882; A61K 2800/4324

(57) ABSTRACT

A composition for coloring hair is disclosed. The composition comprises, in a cosmetically acceptable solvent, at least one reducing agent with a concentration up to 2 wt. % of the composition, preferably up to 1 wt. %. Optionally, the composition comprises at least one coloring agent. The composition is free of ammonia and free of p-phenylenediamine (PPD). The mixing of the composition for coloring hair with an oxidizing composition launches an exothermic chemical reaction providing heat with a temperature that increases from room temperature TR to a temperature ranging from about (T_{R}+3)° C. to about (T_{R}+20)° C. A kit and a method for changing hair color are also disclosed. The compositions and methods are achieving color change results and white hair coverage with larger dye molecules than PPD, in an ammonia free environment. The exothermic properties allow obtaining uniform and rich hair colors in without ammonia or PPD.

### CROSS-REFERENCE TO RELATED APPLICATIONS

The present patent application claims the benefits of priority of commonly assigned U.S. Patent Application No. 61/940,063 entitled “TWO-PART EXOTHERMIC HAIR COLOR COMPOSITION” and filed at the United States Patent and Trademark Office on Feb. 14, 2014.

### FIELD OF THE INVENTION

The present invention belongs to the field of hair care, and more particularly to the field of hair color change. More particularly, the invention is directed to an exothermic composition for changing hair color. The present invention is also directed to a method for changing hair color by using the composition in combination with an oxidizing composition, and kits for the commercialization of the composition.

### BACKGROUND OF THE INVENTION

Conventional methods of permanently modifying the color of hair and eyebrows usually consists in mixing a dye composition containing a mixture of oxidative dye precursors and/or direct dyes, along with an alkalizing agent such as ammonia, with an oxidizing composition containing an oxidizing agent, usually hydrogen peroxide. The two compositions are mixed together prior to use and immediately applied to hair for a certain period of time and then rinsed.

Natural hair pigmentation is determined by melanin, a protein of high molecular weight and very low chemical reactivity. In order to efficiently alter melanin pigment, hair fibers must be treated under high oxidation and alkaline medium. Under such conditions, hair may loose its natural fatty compounds resulting in loss of sheen, dryness and ultimately breakage.

The depth of a shade corresponds to the intensity of the color and can be obtained by varying the concentration of dyes intermediates and/or direct dyes, while the tone can be adjusted by the combination between primary intermediates and couplers. Different shades are designated with a letter and a number. The letter describes the tone (N for natural or neutral, R for red, A for ash, G for gold), while the number refers to the depth of the shade, also called the level of the shade. Shade symbols and corresponding definitions are listed in the Table S.

TABLE S
Shade symbols and definitions:

| Shade | | Shade | |
|---|---|---|---|
| symbols | Shades | symbols | Shades |
| N1 | Black | N6 | Dark Blond |
| N2 | Very Dark | N7 | Medium Blond |
| | Brown | | |
| N3 | Dark Brown | N8 | Light Blond |
| N4 | Medium Brown | N9 | Very Light Blond |
| N5 | Light Brown | N10 | Very Pale Blond |

| | |
|---|---|
| 12 series | Extra Blondes |

Ammonia is the most common alkali agent used in the hair color industry due to its ability to lighten melanin pigment, dye un-pigmented hair fibers (white hair) and shade durability Ammonia increases hair porosity and is extremely irritant to the skin, eyes and respiratory system. Furthermore, ammonia volatile nature and pungent odor causes difficulty with manufacturing process and instability in shade results.

Many attempts to replace ammonia have been made, however this alternative has its limitations Ammonia formulations are able to lighten up to 5 levels whereas any other formulation, free of ammonia, is able to lighten only up to 3 levels. The successful results obtained by ammonia hair color systems are due to powerful oxidative species formed upon reaction of hydrogen peroxide with ammonia (perhydroxy anions). These oxidative species under high pH efficiently lightens keratin fibers.

The drawback of such system relies on the volatile nature of ammonia that causes unpleasant odor, eyes irritation, respiratory diseases and unpredictable results in regards to coloring of keratin fibers. Furthermore, manufacturing the ammonia system requires specific equipment and gases in order to control volatility.

The most common alkali agent used to replace ammonia is ethanolamine or monoethanolamine (MEA). MEA is normally part of the dyes composition and is able to maintain high pH once the second composition containing an acid hydrogen peroxide solution is incorporated to the mixture. However, the oxidative species formed by MEA and hydrogen peroxide are not sufficient to efficiently lighten keratin fibers, such as the 5 levels obtained by ammonia systems. Increasing MEA concentration cannot solve this lack of strength, because skin irritation and severe dermatitis may occur.

Furthermore, plenty of hair color compositions that do not contain ammonia require heat appliances, such as hair dryers or drying helmets, in order to achieve optimum results. It is known that temperatures above 60° C. can burn the scalp. Heat appliances are most likely to be designed with a safe cut-off switch to avoid higher temperature.

Unfortunately, the heat settings of such appliances take in consideration skin sensitivity and not hair itself. Exposure to extreme heat, under alkaline conditions, reduces the hair natural moisture content below the normal level causing dryness and breakage.

It is well known in the art that the use of heat sources increases the brightness of the hair. Free ammonia coloring systems of the art may not involve such exothermic properties and therefore, it is necessary for prior art methods to use heat generated by electrical appliances in order to obtain equivalent hair color modifications of the present invention.

Applicant has previously developed and successfully commercialized a three-phase exothermic composition and method for coloring hair (U.S. Pat. Nos. 7,806,939 B2 and 7,879,114; Canadian patent CA 2 573 567 C). The first phase contains a reducing agent and optionally a coloring agent, the second phase contains an alkalizing agent, and the third phase contains an oxidizing agent. The hair dresser can choose the force of the alkalizing phase between mild, medium and strong force thereof, in accordance with the type of hair of the person.

Applicant has also previously developed a two-phase formulae and method for changing hair color (WO 2010/135827 A1—Cohen et al.). The first phase contains a reducing agent with a strong concentration of reducing agent superior to 1 wt. % and more particularly superior to 2 wt. %.

Although the formulae of the prior art are very efficient, they contain an amount of reducing agent superior to 1 wt. %, preferably superior to 2 wt. %, and p-phenylenediamine (PPD) which has been widely used until now as a permanent hair dye. The use of PPD as a hair dye was very popular because it is a primary dye intermediate with the smallest molecular weight, which means PPD has a better penetration of the dye into the hair cortex. Hair can also be shampooed without becoming discolored and perming to achieve waves or curls can be done without difficulty. However, severe allergy to PPD may occur, resulting in contact rash (urticaria) and rarely anaphylaxis. Also, people working with PPD such as hairdressers, may develop dermatitis on their hands.

Hence, in light of the aforementioned, there is a need for a new hair coloration or decoloration system, which by virtue of its design and components, would be able to overcome some and preferably all of the aforementioned prior art problems.

### SUMMARY OF THE INVENTION

One object of the present invention is to provide a composition for coloring hair. The composition comprises, in a cosmetically acceptable solvent, at least one reducing agent with a concentration up to 2 wt. % of the composition, preferably up to 1 wt. %, more preferably from 0.3 to 1 wt. % of the composition. Optionally, the composition comprises at least one coloring agent. The composition of the invention is free of ammonia and free of p-phenylenediamine (PPD). The mixing of the composition for coloring hair with an oxidizing composition launches an exothermic chemical reaction providing heat with a temperature that increases from room temperature T_{R} to a temperature ranging from about (T_{R}+3)° C. to about (T_{R}+20)° C.

Another aspect of the invention concerns a kit for commercializing the composition for coloring hair as defined herein. The kit comprises:
a first container comprising the composition for coloring hair; and
a manual of instructions for using said composition for coloring hair in combination with an oxidizing composition.

Another aspect of the invention concerns a method for modifying hair color. The method comprises the steps of:

providing a composition for coloring hair, said composition comprising in a cosmetically acceptable solvent, at least one reducing agent with a concentration up to 2 wt. % of the composition, preferably up to 1 wt. %, more preferably from 0.3 to 1 wt. % of the composition; and optionally at least one coloring agent, the composition being free of ammonia and p-phenylenediamine;

providing an oxidising composition comprising in an cosmetically acceptable solvent, at least one oxidising agent with a concentration ranging from about 1 wt. % to 15 wt. % of said oxidizing composition;

mixing the composition for coloring hair and the oxidising composition at room temperature (T_{R}) for obtaining a mixture, said mixing launching an exothermic chemical reaction providing heat with a temperature that increases from room temperature T_{R} to a temperature ranging from about (T_{R}+3)° C. to about (T_{R}+20)° C., without the use of external heating; and

applying said mixture to hair immediately after the mixing and for a suitable period of time for modifying hair color.

In the composition and method disclosed herein, reducing agent(s) may be sodium metabisulfite, ammonium thioglycolate, thiolactic acid, cysteamine, cysteine, cystamine, glycerol monothioglycolate, thioglycerol, sodium hydrosulfide or a mixture thereof.

In the composition and method disclosed herein, coloring agent(s) comprises dye intermediates, direct dyes or mixtures thereof.

The composition for coloring hair disclosed herein may further comprise at least one alkalizing agent with a concentration up to 10 wt. % of the hair coloring composition. Alkalizing agent(s) may be monoethanolamine, triethanolamine, ammonium hydroxide, sodium hydroxide, potassium hydroxide, calcium hydroxide, aminopropanol amine, monoisopropanolamine, aminoethylpropanol, guanidine carbonate, sodium silicate, sodium carbonate or mixtures thereof.

The composition for coloring hair disclosed herein may further comprise cosmetically acceptable components or excipients selected from the group consisting of pH-modifiers, viscosity modifiers, thickeners, other solvents, perfumes, colorants, opacifiers, surfactants, preservatives, emulsifiers, stabilizers and mixture thereof.

In the composition and method disclosed herein, the oxidizing composition to be mixed with said composition for launching the exothermic chemical reaction may comprise an oxidising agent in a cosmetically acceptable solvent. The oxidising agent may be hydrogen peroxide, oxygen peroxide, persulfate salts, bromate, sodium perborate, sodium carbonate peroxide or mixture thereof. Preferably, the oxidising agent is hydrogen peroxide. The oxidising agent may be present in the oxidising composition in an amount ranging from about 1 wt. % to 15 wt. % of the oxidizing composition.

In the method disclosed herein, the concentration of the at least one oxidizing agent is preferably provided according to the new hair color wanted by a person and a type of hair of said person to be colored.

In the method disclosed herein, the suitable period of time varies from 10 to 75 minutes.

As mentioned herein, the presence of a coloring agent within the reducing composition is optional. In that connection, it is to be understood by those skilled in the art that when the composition does not contain a coloring agent, the use of the composition as defined herein, or the application of the method as defined herein, will result in a discoloration of hair, corresponding to a depigmentation.

It is also to be understood by those skilled in the art that when the composition for coloring hair contains a coloring compound or agent, the use of the compositions as defined herein, or the application of the method as defined herein, will result in simultaneous action of discoloration—coloration of the hair, corresponding to a depigmentation—pigmentation of the hair.

It will be understood by those skilled in the art that the concentrations of suitable reducing, alkalizing and/or coloring agents in the composition for coloring hair, or the concentration of suitable oxidizing agent in the oxidizing composition, may vary depending on, for example, the type of hair to be treated, the intensity of the shade and color result desired.

Once the reducing and oxidizing compounds of the two compositions are mixed together, prior to application of the mixture on hair, the resulting mixture generates a certain amount of heat during a certain amount of time. The exothermic reaction is due to a chemical reaction between the reducing and oxidizing agents. The amount of self-generated heat and the heating time are controlled by the concentrations of the reducing agent and/or oxidizing agent.

Composition(s) and method(s) of the present invention have the property, among others, to:
demi-permanently (wash off after several washes) or permanently change the color of hair; and
cover white hair with a high efficiency.

The compositions and method of the present invention are achieving color change results and white hair coverage with larger dye molecules than PPD, in an ammonia free environment. It is believed this increased performance is achieved thanks to the exothermic reaction.

The exothermic properties of the mixture disclose herein also allow for an improved coloring agent penetration into hair, and thus improve aesthetic properties such as conditioning and final shine.

The color change results and white hair coverage are perfectly achieved according to a synergetic combination of:
specific concentration(s) of reducing agent(s) present(s) in the hair color composition allowing exothermic reaction with specific concentrations of oxidizing agents of the oxidizing composition;
a reducing hair color composition free of ammonia;
a reducing hair color composition free of PPD; and
a controlled exothermic reaction of the mixture resulting of the mixing of the reducing and oxidizing compositions.

The exothermic properties of the present invention also allow a user to obtain a uniform and rich hair color comparatively to the prior art in the absence of ammonia and PPD.

The objects, advantages and other features of the present invention will be better understood upon reading of the following non-restrictive description of preferred embodiments thereof, given for the purpose of exemplification only.

### BRIEF DESCRIPTION OF THE DRAWINGS

The above and other objects, features and advantages of the invention will become more readily apparent from the following description, reference being made to the accompanying drawings in which:

FIG. 1 is a graphic presenting temperature versus time for the same shade activated with different concentrations of H₂O₂, in accordance with a preferred embodiment of the invention.

### DETAILED DESCRIPTION OF THE PREFERRED EMBODIMENT

A novel exothermic hair color composition will be described hereinafter. Although the invention is described in terms of specific illustrative embodiment(s), it is to be understood that the embodiment(s) described herein are by way of example only and that the scope of the invention is not intended to be limited thereby.

The terminology used herein is in accordance with definitions set out below.

As used herein % or wt. % means weight % unless otherwise indicated. When used herein % refers to weight % as compared to the total weight percent of the phase or composition that is being discussed.

By “about”, it is meant that the value of weight %, time, pH or temperature can vary within a certain range depending on the margin of error of the method or device used to evaluate such weight %, time, pH or temperature. A margin of error of 10% is generally accepted.

By “room temperature” (T_{R}), it is meant the temperature where the compositions have been stored and said mixture prepared. A room temperature of between about 15 and 30° C. is generally accepted, preferably between about 20 and 26° C.

By “hair”, it is meant any sort of keratinous fibers on the head, on the face such as eyelashes, eyebrows, beard hairs, and on the rest of the body such as body hair or pubic hair.

The term “cosmetically acceptable carrier”, “cosmetically acceptable excipient”, or “cosmetically acceptable solvent”, includes any and all solvents, excipients, carriers or dispersion media, known in the art, that are cosmetically or dermatologically acceptable. The use of such media and agents for cosmetically active substances is well known in the art. Except insofar as any conventional media or agent is incompatible with the active ingredient, its use in the cosmetic compositions is contemplated. Supplementary active ingredients can also be incorporated into the compositions.

The compositions, kits and methods of the present invention are suited to be used to modify the color of different types of hair. By “types of hair” it is meant:
hair having different textures which may depend on the ethnic origins of the person such as, but not limited to Asian, Caucasian, Latino, or African hair;
natural hair color such as black, brown or blond (see Table S for different shades);
grey or white hair; and/or
pre-treated hair (artificial color, bleached, straightened or waved hair).

By “chemically treated hair” it is meant hair that have been previously treated before being further treated with the method as defined herein. For example, hair that have been previously permanently waved, straightened, colored or bleached.

By “permanent” hair color change it is meant that the new hair color, after the use of the compositions or method as defined herein, is permanent or substantially resistant to washout.

By “demi-permanent” hair color change it is meant that the new hair color, after the use of the compositions or method as defined herein, is temporarily and washes off after several shampoos.

By “shade”, it is meant the color imparted to the hair by a mixture of dyes intermediates and/or direct dyes. The different shades obtained from a combination of dyes intermediates and/or direct dyes can be described in terms of the “depth of the shade” (intensity) and the “tone” (color).

### Compositions for Coloring Hair

It is disclosed a composition for changing hair color, free of ammonia and PPD, which has to be mixed with an oxidizing composition before applying the mixture to hair.

As aforesaid, the reducing composition is free of ammonia or ammonium hydroxide. Ammonia is the most common alkali agent used in the hair industry due to its ability to lighten melanin pigment, dye un-pigmented hair fibers (white hair) and shade durability. Ammonia is known to increase hair porosity and to be extremely irritant to the skin, eyes and respiratory system. Furthermore, ammonia volatile nature and pungent odor causes difficulty with manufacturing process and instability in shade results.

### Reducing Agents:

The composition comprises at least one reducing agent. The reducing agent(s) may be a thio compound such as sodium metabisulfite, ammonium thioglycolate, thiolactic acid, cysteamine, cysteine, cystamine, glycerol monothioglycolate, thioglycerol, sodium hydrosulfide or a mixture thereof. More preferably, the reducing agent(s) may be sodium metabisulfite. Sodium metabisulfite or sodium pyrosulfite (American spelling), or sodium metabisulphite or sodium pyrosulphite (English spelling) is an inorganic compound of chemical formula Na₂S₂O₅. The name is sometimes referred to as disodium metabisulfite.

The concentration of reducing agent in the reducing composition is adapted or varies according to the percentage and/or nature of the coloring agent(s) present in the same reducing composition. The concentration up to about 2 wt. % of the reducing composition; preferably with a concentration up to about 1 wt. % of the composition, more preferably with a concentration between 0.3 and 1 wt. % of the reducing composition. According to another aspect of the invention, the concentration of reducing agent is kept below 1 wt. %.

It is further to be understood that the reducing agent(s) provided for the making of the composition, when in contact with oxidizing agent(s) such as hydrogen peroxide, will produce heat produced by an exothermic chemical reaction between these two agents.

### Alkalizing Agents

The composition as defined herein may also comprise at least one alkalizing agent. The alkalizing agent may be an amino base such as 2-aminoethanol, also named ethanolamine or monoethanolamine (MEA), thethanolamine, ammonium sodium, potassium or calcium hydroxide, aminopropanol amine, monoisopropanolamine, aminoethylpropanol, guanidine carbonate, sodium silicate, sodium carbonate or mixtures thereof. More preferably, the alkalizing agent is monoethanolamine (MEA).

It is to be understood that the hair color composition has a pH that may depend on the amount of alkalizing compound used in the first phase. For instance, the pH of the hair color composition may be in a range of from about 4.5 to about 11.0.

### Colorings Agents

The composition for changing hair color as defined herein, optionally comprises a coloring agent.

As known in the art of coloring hair, permanent and demi-permanent systems, both uses oxidative dyes, whereas semi-permanent systems use non-oxidative dyes. The present invention can be used with these three different systems.

As aforesaid, the composition does not contain a coloring agent, the use of the composition as defined herein, or the application of the method as defined herein, will result in a discoloration of hair, corresponding to a depigmentation. In this case, the discoloration of the hair will occur only if time and concentration of oxidizer are elevated (see examples 5 and 6). Otherwise, the choice and concentrations of coloring agents vary depending on, for example, the intensity of the desired color. Preferably, the concentration of the coloring agent is equal or lower than 7 wt. %.

Oxidative and/or direct coloring agents are used in combination with the oxidizing agent to deliver permanent or demi-permanent results to the hair. The coloring agents may be selected from the group of dyes intermediates, direct dyes and mixtures thereof.

The oxidation dye intermediates, or dye intermediates, used in oxidative dyes may be aromatic diamines, naphthols, phenols, aminophenols and their derivatives.

These dye intermediates can be classified as primary and secondary intermediates. Primary intermediates are chemical compounds, which by themselves will form a dye upon oxidation. The secondary intermediates, also known as color modifiers or couplers, are used with other intermediates for specific color effects or to stabilize the color.

The dye intermediates of the composition, the method or the kit, may comprise aromatic compounds such as aromatic diamines, naphthols, polyhydric phenols, aminophenols, derivatives thereof or mixtures thereof. Aromatic compounds derivatives are for example N-substituted derivatives of the amines, ethers of the phenols, or the like.

Dye intermediates are generally colorless molecules prior to oxidation. The oxidation dye color is generated when the primary intermediate is “activated” and subsequently joined with a secondary intermediate (coupling agent), which is also generally colorless, to form a colored, conjugated molecule. In general terms, oxidation hair dye precursors or intermediates include those monomeric materials which form on oxidation oligomers or polymers having extended conjugated systems of electrons in their molecular structure. Because of this new electronic structure, the resultant oligomers and polymers exhibit a shift in their electronic spectra to the visible range and appear colored.

Color modifiers or couplers, such as those detailed hereinafter, may be used in conjunction with the oxidation dye precursors herein and are thought to interpose themselves in the colored polymers during their formation and to cause shifts in the electronic absorption spectra thereof, thereby resulting in slight color changes. A representative list of oxidation dye precursors suitable for use herein is found in “Bleaches, hair coloring and dye removers” by Florence E. Wall, in “Cosmetic Science and Technology”, Wiley Interscience, Second Edition, Volume 2, Chapter 23; pages 308 to 310.

It is to be understood that the oxidizing agents defined herein are suitable for use (in combination with a source of peroxide as detailed herein) with all manner of oxidation dye precursors and color modifiers and that the precursors detailed below are only by way of example and are not intended to limit the compositions and processes herein.

The typical aromatic diamines, polyhydric phenols, aminophenols, and derivatives thereof, described above as primary dye precursors can also have additional substituents on the aromatic ring, e.g. halogen, alkyl, alkyl substituted additional substituents on the amino nitrogen and on the phenolic oxygen, e.g. substituted and unsubstituted alkyl and aryl groups.

The composition of the present invention may, in addition to the essential oxidative hair-coloring agents, optionally include non-oxidative and other dye materials. Optional non-oxidative and other dyes suitable for use in the hair coloring compositions and processes according to the present invention include semi-permanent, temporary and other dyes. Non-oxidative dyes as defined herein include the so-called “direct dyes”, metallic dyes, metal chelate dyes, fiber reactive dyes and other synthetic and natural dyes as detailed in:
“Chemical and Physical Behavior of Human Hair” by Clarence Robbins, Third Edition, Springer-Verlag, pages 250 to 259;
“Hair coloring products” by Gus S. Kass, in “The Chemistry and Manufacture of Cosmetics”, published by Continental Press, Second Edition, Volume IV, chapter 45, pages 841-920;
“Bleaches, hair coloring and dye removers” by Florence E. Wall, in “Cosmetic Science and Technology”, Wiley Interscience, Second Edition, Volume 2, Chapter 23; pages 279 to 343;
“The Science of Hair Care” edited by C. Zviak, Chapter 7 (pages 235 to 261) and Chapter 8 (pages 263 to 273); and
“Hair Dyes” by J. C. Johnson, Noyes Data Corporation (Park Ridge, New Jersey, U. S. A.) 1973, pages 3-91 and 113-139.

Specific hair dyes which may be included in the reducing composition comprise primary intermediate such as 3-methyl-p-aminophenol; 2,3-dimethyl-p-aminophenol; p-toluenediamine; 2-chloro-p-phenylenediamine; N-phenyl-p-phenylenediamine; N-2-methoxyethyl-p-phenylene-diamine; N,N-bis-(hydroxyethyl)-p-phenylenediamine; 2-hydroxymethyl-p-phenylenediamine; 2-hydroxyethyl-p-phenylenediamine; 4,4′-diaminodiphenylamine; 2,6-dimethyl-p-phenylenediamine; 2-isopropyl-p-phenylene-diamine; N-(2-hydroxypropyl)-p-phenylenediamine; 2-propyl-p-phenylene-diamine; 1,3-di-(p-N,N-bis-(2-hydroxyethyl)-aminoanilino)-2-propanol; 2-methyl-4-dimethyl-aminoaniline; p-aminophenol; p-methylaminophenol; 2-hydroxymethyl-p-aminophenol; 2-methyl-p-aminophenol; 2-(2-hydroxyethyl- aminomethyl)-p-amino-phenol; 2-methoxy-methylpaminophenol; and 5-amino-salicylic acid; catechol; pyrogallol; o-aminophenol; 2,4-diaminophenol; 2,4,5-trihydroxytoluene; 1,2,4-trihydroxy-benzene; 2-ethylamino-p-cresol; 2,3-dihydroxynaphthalene; 5-methyl-o-aminophenol; 6-methyl-o-aminophenol; 2-amino-5-acetaminophenol; 2,5-diaminotoluene; 2-dimethylamino-5-aminopyridine; tetraaminopyrimidine; 4,5-diamino-1-methylpyrazole; 4,5-diamino-1-hydroxyethyl-pyrazole, 6-methoxy-8-aminoquinoline; 2,6-dihydroxy-4-methylpyridine; 5-hydroxy-1,4-benzodioxane; 3,4-methylenedioxyphenol; 4-hydroxyethylamino-1,2-methylene-dioxybenzene; 5-chloro-2,3-dihydroxypyridine; 2-hydroxyethylamino-6-methoxy-3-amino-pyridine; 3,4-methylenedioxyaniline; 7-hydroxy-indole; 5-hydroxyindole; 2-bromo-4,5-methylenedioxyphenol; 3-amino-2-methylamino-6methoxypyridine; 2-amino-3-hydroxypyridine; 4-hydroxy-2,5,6-triamino-pyrimidine, 5-hydroxy-indoline, 7-hydroxyindoline or combinations thereof.

The coupler or secondary intermediate may be utilized to expand the color range by copolymerization with the primary intermediate. These materials can also accelerate color formation.

Specific hair dye intermediates that can be used as couplers in the present invention include but not limited to 4-amino-m-cresol, 2-amino-4-hydroxy-ethylaminoanisole, 4-amino-2-hydroxy-toluene, 4-amino-3-nitrophenol, m-aminophenol, 2-chloro-p-phenylenediamine, 4-chlororesorcinol, 2,4-diaminophenoxy-ethanol, 2-methyl-resorcinol, 1-naphthol, 3-nitro-p-hydroxyethyl-aminophenol, 4-nitro-o-phenylene-diamine, 2-nitro-p-phenylene-diamine, phenyl-methylpyrazolone, m-phenylene-diamine, resorcinol, 2-methyl-5-hydroxyethyl-aminophenol or mixtures thereof.

It should be understood that the descriptions of primary intermediates and couplers given herein are meant to implicitly include the salt forms of those dye molecules that form stable salts. For example, the hydrochloride or sulfate salts in the case of amines, and the alkali metal salts in the case of phenols.

Direct dyes are colored compounds that can be used to modify the color of a substrate by dispersion. Direct dyes are used along with the dyes intermediates to add vibrancy to the tone which is not otherwise available if the composition only contains oxidation dye intermediates. The dye composition disclosed herein may also comprise at least one direct dye that may be chosen, for example, from nitrobenzene dyes, cationic direct dyes, azo direct dyes and methine direct dyes.

The direct dyes may also be chosen in a group of compounds containing 2-amino-6-chloro-4-nitrophenol (B99, or RODOL 9R (Lowenstein), or Chlororange Base (Wella)), Basic Blue #3, External D & C Violet #2, Basic Green #4, Basic Orange #1, Basic Red #22, Red #2, Basic Red #46, Basic Violet #13, Basic Violet #1, Basic Yellow #11, Basic Yellow #28, Basic Brown #16, Basic Brown #17, Basic Blue #99, Blue #2, Red #13, basic yellow 57, basic yellow 40, basic yellow 87, basic blue 26, basic green 4, basic orange 2, basic red 51, basic blue 77 and mixture thereof.

### Other Components of the Hair Color Compositions

The composition of the invention may further contain a plurality of components, carriers or excipients well known in the art of hair color formulation such as: pH-modifiers, viscosity modifiers, thickeners, solvents (alcohol, aqua, etc), perfumes or fragrances, colorants (for coloring the formulations, not the hair), opacifiers, surfactants (ionic or non-ionic such as zwitterionic), preservatives, emulsifiers, stabilizers or the like. Deionized water may be used in the compositions (see examples).

### Oxidizing Compositions

The composition for coloring hair of the invention has to be mixed with an oxidizing composition containing at least one oxidizing agent, also named oxidizer. The amount of the oxidizing agent in the oxidizing composition may be from about 1 wt. % to about 15 wt. % of the total weight of the oxidizing composition.

The oxidizer may be a peroxide derivative such as hydrogen or oxygen peroxide, a persulfate salts, bromate, sodium perborate, sodium carbonate peroxide or mixture thereof. More preferably, the oxidizing compound is hydrogen peroxide (H₂O₂) presents in hydrogen peroxide compositions.

As known in the art of hair coloration, the unit for measuring the concentration of hydrogen peroxide is the Volume (Vol.). The volume of a hydrogen peroxide solution is the number of liters of oxygen in its gaseous form released by the decomposition taking place in a liter of that particular hydrogen peroxide solution. The different volumes of H₂O₂ being part of the oxidizing composition may be ranging from about 3 to 50 Vol., corresponding approximately to a concentration ranging from about 1 wt. % to 15 wt. % of the oxidizing composition.

The oxidizing composition may further contain a plurality of components and excipients well known in the art of hair color formulation, such as those already disclosed herein above for the hair color composition.

### Methods for Changing Hair Color

As aforesaid, another aspect of the invention concerns a method for modifying hair color. The method comprises several steps performed by a hair dresser, a hair colorist, or the person who wants his or her hair to be colored or discolored.

A step consists in providing or selecting a composition for changing hair color according to the present invention, and as defined or claimed herein.

Another step consists in providing or selecting the adequate oxidising composition as defined or claimed herein.

The choice of the composition's components is made in accordance of several parameters including, but limited to, the shade of hair to be modified, the final color desired, the type of hair, the presence of grey hair to be covered, etc.

Another step of the method consists in mixing the composition for changing hair and the oxidising composition together, using tools and recipients known in the art. The mixing is generally performed at room or ambient temperature: T_{R}. A mixture is obtained and immediately applied to the hair.

As known in the art of hair coloration, the parts or amounts of hair color and oxidizing compositions are weighted by the user, the colorist or the hairdresser, before being mixed. In the method defined herein, a first part or amount of hair color composition and a second part or amount of the oxidizing composition are mixed with a ratio [first amount]/[second amount] from 1/1 to 1/2, preferably with a ratio being 1/1, 1/1.5 or 1/2. The ratios are selected to make the mixing easier for stylists. Needless to say that formulas can be adjusted to any maxing ratio, including 1:1.5 by calibrate the concentration of chemical compounds.

As previously said, reducing agents(s) and oxidizing agents(s) when mixed immediately launches an exothermic chemical reaction providing heat, without the use of external heating. The temperature may increase from room temperature T_{R} to a temperature ranging from about (T_{R}+3)° C. to about (T_{R}+20)° C. Precaution is taken than the temperature is not too high for not damaging the hair and scalp of the person.

The temperature of the resulting mixture T increases from room temperature T_{R} until to reach a quite constant value or plateau for a variable period of time. The increase of temperature T and the duration of the plateau vary depending on the concentration and amount of active agents present in the first and second phase, such as reducing and oxidizing agents, which as aforesaid, depend on the type, amount and original color of hair to be treated and also depend on the final desired shade. The heat effect lasts throughout the coloring process; up to about 70 minutes.

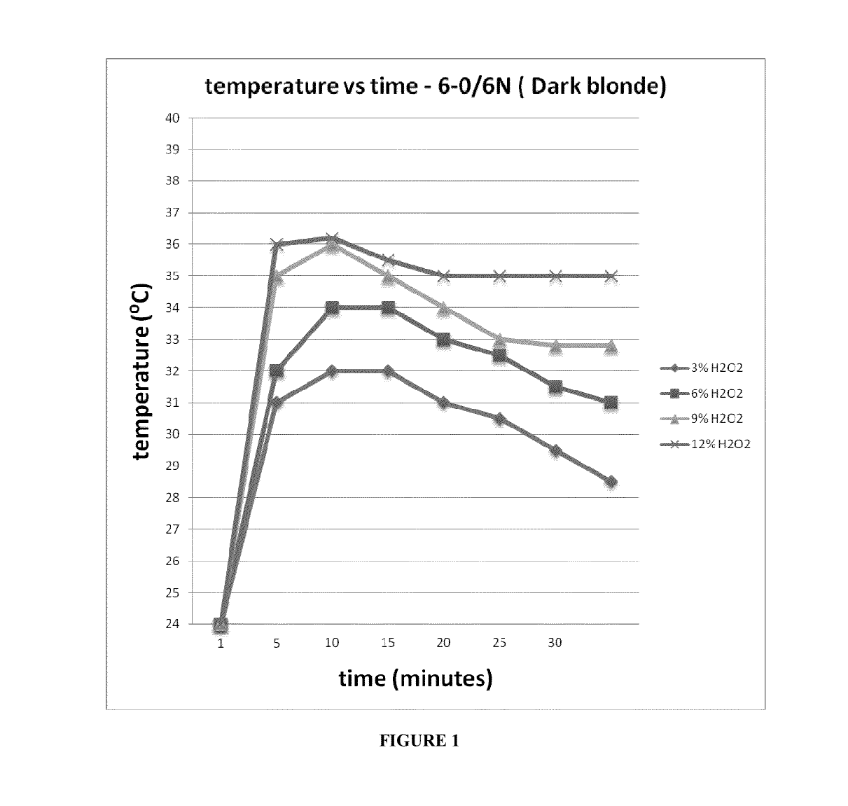

FIG. 1 illustrates the variation in time of temperature of different coloring systems. Depending on the degree of lightening desired, the concentration of H₂O₂ is increased, resulting to higher heat produced. Also, it allows maintaining the temperature at an elevated plateau. The boost in temperature favors the chemical activity enhancing the simultaneous process of lightening the keratin fibers and dyes penetration.

Therefore, the period of time of the method during which the mixture is applied to hair may also vary according to the type, amount and original color of hair to be treated. It will also depend on the final desired shade.

Heat from the exothermic reaction increases the bleaching performance of the oxidizing agent, such as a peroxide derivative, under alkaline conditions. Moderated heat is preferably advised in order to achieve lightening of hair melanin without damaging the hair fibers.

The mixture is kept on hair for a suitable period of time for modifying hair color. Then the hair are washed and rinsed to remove the mixture from the hair.

The method of the invention may include other steps well known in the art for changing the color of keratinous fiber.

### Kits

The present invention further concerns different kits for the distribution or commercialization of the compositions of the invention.

According to a first aspect of the invention, kit(s) may comprise at least two containers, the first container comprising the hair color composition as defined herein, and the second container comprising the oxidizing composition as defined herein. Optionally, the kit(s) may include a manual of instructions to assist the user, stylist, colorist or hairdresser to perform the method disclosed herein. The manual can be in a form of a book provided separately for different color compositions. The instructions can also be directly written on a box containing the compositions. The manual may also disclose the method of the present invention as disclosed herein.

According to another aspect of the invention, kit(s) may comprise a container comprising the hair color reducing composition as defined herein, and the manual of instructions as disclosed above, for using the composition in combination with an oxidizing composition. In that case, the oxidizing composition may be sold or commercialized as part of the kit or separately.

Containers of the kits may be in a form of a tube, a bottle, a pot or any sort of containers generally used in commercialization and distribution of cosmetic products.

Kit(s) of the invention may further comprise any tools that may be helpful for the mixing of the two phases or compositions, and the application of the resulting mixture to hair. These tools may include a graduated tube for measuring the volume of each composition to mix, a mixing pot for mixing them, a brush for applying the mixture on hair, additional containers comprising shampoo and/or hair conditioner for rinsing, washing hair, and/or protective gloves.

Each member of the kit may be packed into a box, a bag or any kind of known recipient used in the commercialization and distribution of cosmetic products.

### EXAMPLES

The following examples illustrate some preferred embodiments of the invention. Tables A and B below respectively disclose basic ingredients preferably used for the formulation of hair color composition(s) (the color) and oxidizing composition(s).

TABLE A
Examples of hair color compositions

| | | Concentrations |
|---|---|---|
| Generic names | Components | (wt. %) |
| Reducing Agent | Sodium metabisulfite | 0.3-2.0 |
| Alkalizing Agent | Ethanolamine | 0-10.0 |
| Coloring Compounds | Dyes intermediates | 0-6.0 |
| | Direct dyes | 0-1.0 |
| Antioxidant | Sodium sulfite | 0-1.0 |
| Chelating Agent | Trisodium HEDTA | 0.35 |
| Antioxidant | Sodium Isoascorbate | 0-1.0 |
| Solvent | Propylene glycol | 2.0 |
| Solvent | Ethoxydiglycol | 2.5 |
| Thickener | Xanthan gum | 0.14 |
| Thickener | Stearyl alcohol | 4.0 |
| Thickener | Cetyl alcohol | 4.0 |
| Emulsifier | Glyceryl stearate | 4.0 |
| Non-Ionic Surfactant | Oleyl alcohol | 1.0 |
| Amphoteric Surfactant | Cocamidopropylbetaine | 1.0 |
| Rheology Modifier | Carbomer | 0-1.0 |
| Non-Ionic Surfactant | Polysorbate 80 | 0.5 |
| Preservative | DMDM Hydantoin | 0.4 |
| Solvent | Deionized water | q.s. to 100 |

TABLE B
Examples of oxidizing compositions

| | | Concentrations |
|---|---|---|
| Generic Names | Components | (wt. %) |
| Oxidizer | Hydrogen peroxide | 1-15 |
| Stabilizer | Sodium stannate | 0.03 |
| Opacifier | Cetyl alcohol | 1.0 |
| Thickener | Stearyl alcohol | 1.0 |
| Emulsifier | Ceteareth 5 or 20 | 0.5 |
| Surfactant | Sodium lauryl sulfate | 1.0 |
| Ph Adjuster/Stabilizer | Phosphoric Acid or | 0.5 |
| | Etidronic Acid | |
| Solvent | Deionized water | q.s. to 100 |

Concentrations of the components written in bold Tables A and B are variable. Concentrations in the formulations will depend on different parameters such as the type of hair of the person, the desired final hair color, etc.

In the following examples, we refer to Table S for the meanings of the shade symbols N1 to N10.

### Example 1

### From Virgin Natural Medium Brown Hair (N4) to Dark Brown Hair (N3)

TABLE 1
hair color composition of example 1
Quantity (wt. %) of:
Dyes

| | |
|---|---|
| Toluene-2,5-Diamine Sulfate | 2.3000 |
| m-Aminophenol | 0.065 |
| Resorcinol | 0.7780 |
| 2-Methylresorcinol | 0.0760 |
| 4-Chororesorcinol | 0.2836 |

Reducing agent

| | |
|---|---|
| Sodium metabisulfite | 0.3 |

Alkalizing agent

| | |
|---|---|
| Ethanolamine | 6.0 |

No lightening of hair is required. Therefore a low percentage of hydrogen peroxide (3.0 wt. %, or 10 Vol.) in the oxidizing composition is needed to be mixed with the composition of example 1 (Table A combined with Table 1).

The two compositions are then mixed together in a ratio 1:1 and applied to hair for a period of 30 minutes.

An increase of temperature of +5° C.+/−1° C. is obtained from the beginning of processing time (room temperature at 20° C.), to the end.

### Example 2

### From Virgin Natural Medium Brown Hair (N4) to Dark Brown Hair (N3)

TABLE 2
hair color composition of example 2
Quantity (wt. %) of:
Dyes

| | |
|---|---|
| Toluene-2,5-Diamine Sulfate | 2.3000 |
| m-Aminophenol | 0.065 |
| Resorcinol | 0.7780 |
| 2-Methylresorcinol | 0.0760 |
| 4-Chororesorcinol | 0.2836 |

Reducing agent

| | |
|---|---|
| Sodium metabisulfite | 0.3 |

Alkalizing agent

| | |
|---|---|
| Ethanolamine | 4.0 |

No lightening of hair is required. A demi-permanent hair color application is desired. Therefore a low percentage of hydrogen peroxide (3.0 wt. %, or 10 Vol.) in the oxidizing composition is needed to be mixed with the color composition of example 2 (Table A combined with Table 2).

The two compositions are then mixed together in a ratio 1:1 and applied to hair for a period of 20 minutes.

An increase of temperature of +5° C.□1° C. is obtained from the beginning of processing time (room temperature at 20° C.), to the end.

### Example 3

### From Virgin Natural Dark Blonde Hair (N6) to a Natural Very Light Blonde (N9)

TABLE 3
hair color composition of example 3
Quantity (wt. %) of:
Dyes

| | |
|---|---|
| Toluene-2,5-Diamine Silfate | 0.6500 |
| Resorcinol | 0.3200 |
| m-Aminophenol | 0.0070 |

Reducing agent

| | |
|---|---|
| Sodium metabisulfite | 1.0 |

Alkalizing agent

| | |
|---|---|
| Ethanolamine | 7.0 |

Lightening of hair is required in example 3. Therefore a high percentage of hydrogen peroxide (9.0 wt. % or 30 vol.) in the oxidizing composition is needed to be mixed with the hair color composition of example 3 (Table A combined with Table 3).

The two compositions are then mixed together in a ratio 1:1 and applied to the hair for a period of 45 minutes.

An increase of temperature of +10° C.□1° C.is obtained from the beginning of processing time (room temperature at about 20° C.), to the end.

### Example 4

### From a Virgin Natural Dark Blonde Hair (N6) to a Pure Blonde (12 series)

TABLE 4
hair color composition of example 4
Quantity (wt. %) of:
Dyes

| | |
|---|---|
| Toluene-2,5-Diamine Sulfate | 0.4000 |
| Resorcinol | 0.1300 |
| 4-Amino-2-Hydroxytoluene | 0.0618 |
| m-Aminophenol | 0.2075 |

Reducing agent

| | |
|---|---|
| Sodium metabisulfite | 1.5 |

Alkalizing agent

| | |
|---|---|
| Ethanolamine | 10.0 |

Lightening of hair is required. Therefore a high percentage of hydrogen peroxide (12.0 wt. % or 40 vol.) in the oxidizing composition needs to be mixed with the hair color composition of example 4 (Table A combined with Table 4).

The two compositions are then mixed together in a ratio 1:2 and applied to the hair for a period of 60 minutes.

An increase of temperature of +13° C.□□1° C. is obtained from the beginning of processing time (room temperature at about 20° C.), to the end.

### Example 5

### Virgin Dull Natural Medium Brown Hair (N4) to Virgin Shiny Natural Medium Brown Hair

TABLE 5
composition of example 5
Quantity (wt. %) of:
Reducing agent

| | |
|---|---|
| Sodium metabisulfite | 0.3 |

Alkalizing agent

| | |
|---|---|
| Ethanolamine | 4.0 |

Composition does not contain any dye. No lightening of hair is required. Shine is desired. Therefore a low percentage of hydrogen peroxide (1.5 wt. %, or 5 Vol.) in the oxidizing composition is needed to be mixed with the composition of example 5 (Table A combined with Table 5).

The two compositions are then mixed together in a ratio 1:1 and applied to hair for a period of 10 minutes.

An increase of temperature of +2° C.□1° C. is obtained from the beginning of processing time (room temperature at 20° C.), to the end.

### Example 6

### From a Virgin Natural Dark Blonde Hair (N6) to Very Pale Blonde (No Tint)

TABLE 6
ingredients for the reducing composition of example 6
Quantity (wt. %) of:
Reducing agent

| | |
|---|---|
| Sodium metabisulfite | 1.0 |

Alkalizing agent

| | |
|---|---|
| Ethanolamine | 7.0 |

The composition does not contain any dye. Lightening of hair is required without tint or use of bleach compounds. Therefore a high percentage of hydrogen peroxide (12.0 wt. % or 40 vol.) in the oxidizing composition needs to be mixed with the reducing composition (Table A combined with Table 6).

The two compositions are then mixed together in a ratio 1:1 and applied to the hair for a period of 60 minutes.

An increase of temperature of +10° C.±1° C. is obtained from the beginning of processing time (room temperature at about 20° C.), to the end

### Example 7

### From Bleached Hair (Level 8) to a Very Bright Intense Red Color

TABLE 7
hair color composition of example 7
Quantity (wt. %) of:
Dyes

| | |
|---|---|
| Basic red 51 | 0.396 |
| Basic yellow 87 | 0.0198 |

Reducing agent

| | |
|---|---|
| Sodium metabisulfite | 1.0 |

The composition does not contain any alkalizing agent. Lightening of hair is not required. Therefore a low percentage of hydrogen peroxide (1.5 wt. % or 5 vol.) in the oxidizing composition needs to be mixed with the hair color composition (Table A combined with Table 7).

The two compositions are then mixed together in a ratio 1:1 and applied to the hair for a period of 20 minutes.

An increase of temperature of +2° C.±1° C. is obtained from the beginning of processing time (room temperature at about 20° C.), to the end

While illustrative and presently preferred embodiment(s) of the invention have been described in detail hereinabove, it is to be understood that the inventive concepts may be otherwise variously embodied and employed and that the appended claims are intended to be construed to include such variations except insofar as limited by the prior art.

What is claimed is:

1. A composition for coloring hair comprising in a cosmetically acceptable solvent, at least one reducing agent with a concentration up to 2 wt. % of the composition, and optionally at least one coloring agent, said composition being free of ammonia and p-phenylenediamine; and the mixing of said composition with an oxidizing composition launches an exothermic chemical reaction providing heat with a temperature that increases from room temperature T_{R} to a temperature ranging from about (T_{R}+3)° C. to about (T_{R}+20)° C.

2. The composition of claim 1, wherein the concentration of said at least one reducing agent is up to 1 wt. % of the composition.

3. The composition of claim 1, wherein the concentration of said at least one reducing agent is from 0.3 to 1 wt. % of the composition.

4. The composition of claim 1, wherein said at least one reducing agent is sodium metabisulfite, ammonium thioglycolate, thiolactic acid, cysteamine, cysteine, cystamine, glycerol monothioglycolate, thioglycerol, sodium hydrosulfide or a mixture thereof.

5. The composition of claim 1, wherein said at least one coloring agent is present with a concentration up to about 7 wt. % of the composition.

6. The composition of claim 5, wherein said at least one coloring agent comprises dye intermediates, direct dyes or mixtures thereof.

7. The composition of claim 1, further comprising at least one alkalizing agent with a concentration up to 10 wt. % of the hair coloring composition.

8. The composition of claim 7, wherein said at least one alkalizing agent is monoethanolamine, triethanolamine, ammonium hydroxide, sodium hydroxide, potassium hydroxide, calcium hydroxide, aminopropanol amine, monoisopropanolamine, aminoethylpropanol, guanidine carbonate, sodium silicate, sodium carbonate or mixtures thereof.

9. The composition of claim 1, further comprising cosmetically acceptable components or excipients selected from the group consisting of pH-modifiers, viscosity modifiers, thickeners, solvents, perfumes, colorants, opacifiers, surfactants, preservatives, emulsifiers, stabilizers and mixture thereof.

10. The composition of claim 1, wherein the oxidizing composition to be mixed with said composition for launching the exothermic chemical reaction comprises an oxidising agent in a cosmetically acceptable solvent, said oxidising agent being hydrogen peroxide, oxygen peroxide, persulfate salts, bromate, sodium perborate, sodium carbonate peroxide or mixture thereof.

11. The composition of claim 10, wherein said oxidising agent is present in said oxidising composition in an amount ranging from about 1 wt. % to 15 wt. % of said oxidizing composition.

12. A kit for commercializing the composition for coloring hair as defined in claim 1, comprising:
a first container comprising said composition for coloring hair; and
a manual of instructions for using said composition for coloring hair in combination with an oxidizing composition.

13. A method for modifying hair color comprising the steps of:
providing a composition for coloring hair, said composition comprising in a cosmetically acceptable solvent, at least one reducing agent with a concentration up to 2 wt. % of the composition and optionally at least one coloring agent, said composition being free of ammonia and p-phenylenediamine;
providing an oxidising composition comprising in an cosmetically acceptable solvent, at least one oxidising agent with a concentration ranging from about 1 wt. % to 15 wt. % of said oxidizing composition;
mixing the composition for coloring hair and the oxidising composition at room temperature (T_{R}) for obtaining a mixture, said mixing launching an exothermic chemical reaction providing heat with a temperature that increases from room temperature T_{R} to a temperature ranging from about (T_{R}+3)° C. to about (T_{R}+20)° C., without the use of external heating; and
applying said mixture to hair immediately after the mixing and for a suitable period of time for modifying hair color.

14. The method of claim 13, wherein the concentration of said at least one oxidizing agent is provided according to the new hair color wanted by a person and a type of hair of said person to be colored.

15. The method of claim 13, wherein the concentration of said at least one reducing agent is up to 1 wt. % of the composition.

16. The method of claim 13, wherein the concentration of reducing agent is from 0.3 to 1 wt. % of the composition.

17. The method of claim 13, wherein said at least one reducing agent is sodium metabisulfite, ammonium thioglycolate, thiolactic acid, cysteamine, cysteine, cystamine, glycerol monothioglycolate, thioglycerol, sodium hydrosulfide or a mixture thereof.

18. The method of claim 13, wherein said at least one coloring agent is present with a concentration up to about 7 wt. % of the composition.

19. The method of claim 13, wherein said composition for coloring hair further comprises at least one alkalizing agent with a concentration up to 10 wt. % of the hair coloring composition.

20. The method of claim 13, wherein the suitable period of time varies from 10 to 75 minutes.
